(19) United States
(12) Patent Application Publication
(10) Pub. No.: US 2025/0128998 A1
(43) Pub. Date: Apr. 24, 2025

(54) FCC PRODUCT VAPOUR SEPARATION METHOD FOR IMPROVED PRODUCT RECOVERY

(71) Applicant: LUMMUS TECHNOLOGY LLC, Houston, TX (US)

(72) Inventors: Galip H. Guvelioglu, Houston, TX (US); Sumit Mittal, Houston, TX (US); Rama Rao Marri, Houston, TX (US); Justin Breckenridge, Houston, TX (US)

(73) Assignee: LUMMUS TECHNOLOGY LLC, Houston, TX (US)

(21) Appl. No.: 18/917,998

(22) Filed: Oct. 16, 2024

(30) Foreign Application Priority Data
Oct. 19, 2023 (IN) 202321071470

(51) Int. Cl. C07C 7/00; C07C 7/04; C07C 7/09

(52) U.S. Cl. CPC C07C 7/005; C07C 7/04; C07C 7/09

(57) ABSTRACT

Systems and processes for separating a mixture of cracked hydrocarbons. A main fractionator separates the mixture of cracked hydrocarbons into an overheads comprising C1 to C6+ hydrocarbons, a side draw, and a bottoms. An overheads condensation and high-pressure separation system partially condenses the overheads and compresses the uncondensed vapor to produce a compressed gas fraction and a compressed liquids fraction. A first distillation column receives the compressed liquids fraction and separates the compressed liquids fraction into a first overheads vapor and a first bottoms. A second distillation column separates the first bottoms into a second overheads and a second bottoms. An absorber receives and contacts in countercurrent flow a portion of the second bottoms fraction and the side draw liquid fraction with the compressed gas fraction, producing an absorber overheads fraction comprising offgas and an absorber bottoms fraction. Such systems are useful in integrating existing FCC/RFCC units with petrochemical complexes.

### FIELD OF THE DISCLOSURE

Embodiments of the present disclosure generally relate to efficient separation of catalytically cracked hydrocarbons or other reaction products.

### BACKGROUND

Fluid catalytic cracking (FCC) units and residue fluid catalytic cracking (RFCC) units within a refinery complex are generally designed for producing finished products. The finished products often include offgas consisting of lighter hydrocarbons (rich in methane and ethane), olefins grade product mix (containing predominantly ethylene, propylene and butylenes, and possibly including C5 olefins) recovered as a lights fraction, as well as heavier hydrocarbon fractions including gasolines.

One example of a separation system according to conventional industry practice is shown in FIG. 1 (prior art). To simplify the scheme, only major equipment has been shown. Various components of the separation system are illustrated to provide an overall understanding of the system, however one skilled in the art would recognize that a number of components that may be present are not illustrated, such as pumps, compressors, exchangers, valves, bypass lines, control systems, and others.

The separation system includes main fractionator 102A, main fractionator overheads and high-pressure separation system 104A, primary absorber 108A, secondary absorber 108AA, and a fractionation zone including deethanizer 180A and debutanizer 190A. Primary products recovered from the separation system include fuel gas (C1, C2) fraction 140AA, LPG (C3, C4) fraction 150AA, naphtha range fraction 160A, and heavies fraction 170A. Other fractions may be recovered as well, such as various intermediate hydrocarbon cuts (not illustrated) recovered from main fractionator 102A, among others.

A cracked reaction effluent 112 may be fed to the main fractionator 102A for separating the cracked reactor effluent into a bottoms fraction (heavy product fraction) 170A, one or more side draw fractions (192A, 101A), one or more additional side draw liquid fractions (not illustrated), and an overheads vapor 118A. The side draw liquid fraction portion 101A may fed to secondary absorber 108AA, while the overheads vapor 118A may be processed through the fractionator overheads and the high-pressure separation system 104A, which may include coolers, flash drums, compressor, pumps etc. Separation system 104A provides a wild naphtha 122, primary gas (C1-C4 rich) 130A, and liquid intermediate 132A. Streams 122 and 130A are fed to primary absorber 108A, from which fuel gas 140A is recovered at top which is further fed to secondary absorber 108AA for removing C5+ range hydrocarbons. Stream 140AA recovered from secondary absorber 108AA mix with refinery fuel gas header based on the properties and requirement. Side draw 101A from main fractionator 102A is used as absorbing media in secondary absorber 108AA. Bottom liquid 142A from secondary absorber 108AA is recycled back to main fractionator 102A.

Liquid intermediate 132A from system 104A is fed to deethanizer 180A, which removes a C1-C2 fraction and sends remaining liquid 182A to debutanizer 190A. Debutanizer 190A separates liquid 182A into LPG fraction (150AA) and naphtha fraction (160A). A portion 192AA of naphtha fraction 160A is fed to primary absorber 108A for C3, C4 fraction recovery as described above. This stream recirculation rate directly affects the LPG recovery from the system and controls sizing of majority of the equipment. Naphtha liquid 160A is the product for blending into gasoline pool after necessary treatment (such as sulfur removal).

With the use of electrical vehicles gaining momentum, the on-road gasoline demand is expected to slow or decrease significantly in the coming years. Thus, the traditional separation scheme for FCC units may become unsuitable. As the traditional FCC/RFCC units shift away from gasoline production, toward lighter molecules/BTX rich naphtha, the commonly used FCC separation units such as illustrated in FIG. 1 may be unsatisfactory for processing the reactor effluents to end user products.

### SUMMARY OF THE CLAIMED EMBODIMENTS

In one aspect, embodiments herein relate to a hydrocarbon separation system. The hydrocarbon separation system includes a flow line for conveying a cracked reaction effluent comprising a mixture of hydrocarbons, including methane (C1) to heavy (C12+) hydrocarbons, from one or more cracking reactors. The separation system also includes a main fractionator, wherein the main fractionator is configured to receive the mixture of hydrocarbons, and wherein the main fractionator is configured to separate the mixture of hydrocarbons into an overhead vapor comprising C1 to C6+ hydrocarbons, a side draw liquid fraction, and a bottoms product fraction. A main fractionator overheads condensation and high-pressure separation system is configured to partially condense the overheads vapor and compress the uncondensed vapor to produce a compressed gas fraction and a compressed liquids fraction. A demethanizer is configured to receive the compressed liquids fraction and to separate the compressed liquids fraction into a demethanizer overheads vapor fraction, comprising methane rich gas, and a demethanizer bottoms fraction, comprising C2 to C6+ hydrocarbons. A depentanizer is configured to receive the demethanizer bottoms fraction and to separate the demethanizer bottoms fraction into a depentanizer overheads fraction, comprising C2 to C5 olefins, and a depentanizer bottoms fraction, comprising C6+ hydrocarbons. Further, an absorber is configured to receive and contact in countercurrent flow a portion of the depentanizer bottoms fraction and the side draw liquid fraction with the compressed gas fraction, and to produce an absorber overheads fraction comprising methane and an absorber bottoms fraction.

In another aspect, embodiments herein relate to a hydrocarbon separation system including a flow line for conveying a cracked reaction effluent comprising a mixture of hydrocarbons, including methane (C1) to heavy (C12+) hydrocarbons, from one or more cracking reactors. A main fractionator is configured to receive the mixture of hydrocarbons, and the main fractionator is configured to separate the mixture of hydrocarbons into an overhead vapor comprising C1 to C6+ hydrocarbons, a side draw liquid fraction, and a bottoms product fraction. A main fractionator overheads condensation and high-pressure separation system is configured to partially condense the overheads vapor and compress the uncondensed vapor to produce a compressed gas fraction and a compressed liquids fraction. A first distillation column is configured to receive the compressed liquids fraction and to separate the compressed liquids fraction into a first overheads vapor fraction and a first bottoms fraction. Further, a second distillation column is configured to receive the first bottoms fraction and to separate the first bottoms fraction into a second overheads fraction and a second bottoms fraction. The system further includes an absorber configured to receive and contact in countercurrent flow a portion of the second bottoms fraction and the side draw liquid fraction with the compressed gas fraction, and to produce an absorber overheads fraction comprising offgas and an absorber bottoms fraction.

In some embodiments, the first distillation column is sized and configured to operate flexibly as a demethanizer in a first mode of operation and as a deethanizer in a second mode of operation.

The absorber in some embodiments is sized and configured to operate flexibly to recover an offgas comprising methane and essentially no C2 hydrocarbons in a first mode of operation and to recover an offgas comprising methane and C2 hydrocarbons in a second mode of operation.

The second distillation column is sized and configured, in some embodiments, to operate flexibly as a debutanizer in a first mode of operation and as a depentanizer in a second mode of operation.

In some embodiments, the system may further include a flow line for feeding the absorber bottoms fraction to the main fractionator overheads condensation and high-pressure separation system.

The hydrocarbon separation system of some embodiments further includes a flow line for feeding the first overheads vapor fraction to the main fractionator overheads condensation and high-pressure separation system.

The compressed liquid fraction produced in some embodiments comprises a first compressed liquids fraction comprising C4 to C6+ hydrocarbons and a second compressed liquids fraction comprising C1 to C6+ hydrocarbons. In such and similar embodiments, a flow line is provided for feeding the first compressed liquids fraction to the absorber, a flow line is provided for feeding a portion of the second compressed liquids fraction to the first distillation column, and a flow line is provided for feeding a second portion of the compressed liquids fraction as a reflux to the main fractionator.

In some embodiments, the absorber is further configured to produce an absorber side draw fraction and to receive a cooled absorber side draw fraction, the system further comprising a heat exchanger configured to cool the absorber side draw fractions and to produce the cooled absorber side draw fraction.

The system of some embodiments further comprises a heat exchange system, disposed downstream of the main fractionator and upstream of the absorber, the heat exchange system comprising two or more heat exchangers configured to cool the main fractionator side draw and to produce a cooled main fractionator side draw fed to the absorber.

A flow control system is provided in some embodiments, which is configured for diverting a portion of the cooled main fractionator side draw to the main fractionator as a reflux and for feeding a remaining portion of the cooled main fractionator side draw to the absorber.

In some embodiments, a second heat exchange system is provided for cooling the main fractionator bottoms fraction. The heat exchange system and the second heat exchange system, in some embodiments, are configured to heat water via the main fractionator side draw and the main fractionator bottoms fraction to produce a medium or high-pressure steam stream.

Optionally, in some embodiments, at least one of the two or more heat exchangers of the heat exchange system comprises a heat exchanger configured to reboil a bottoms stream recovered from the first distillation column and to return a heated reboil bottoms stream to the first distillation column.

The high-pressure separation system of some embodiments includes: a heat exchanger and condensate drum for partially condensing the overhead vapor to produce a first liquid fraction and an overhead vapor fraction; a first stage compressor configured to compress the overheads vapor fraction and produce a compressed overheads vapor fraction; a separator configured to separate condensates from the compressed overheads vapor fraction and to produce a compressed vapor fraction and a compressed liquid fraction; a second stage compressor configured to further compress the compressed vapor fraction and to produce a second stage compressor effluent; and a high-pressure separator configured to separate liquids and vapors contained in the second stage compressor effluent and the compressed liquids fraction to produce a high-pressure separation system liquid stream (which may be fed to the first distillation column as the compressed liquids fraction) and a high-pressure separation system vapor stream (which may be fed to the as the compressed gas fraction). In some embodiments, a mixing system is configured to mix the second stage compressor effluent with the absorber bottoms stream and the first distillation column overheads vapor fraction.

The system of some embodiments includes a second distillation column overheads system comprising: a heat exchanger for partially condensing the second distillation column overheads fraction; a drum for separating the partially condensed second distillation column overheads fraction into a second distillation column overheads liquid fraction and a second distillation column overheads vapor fraction; a knock-out drum to collect any liquids entrained in the second distillation column overheads vapor fraction, producing a knock-out vapor and a knockout liquid; a compressor for compressing the knock-out vapor, producing a compressed product; a flow line for feeding a portion of the second distillation column overheads liquid fraction to the second distillation column as a reflux; and a flow line for recovering a remaining portion of the second distillation column overheads liquid fraction as a mixed olefin rich product. In some embodiments, a mixing system is provided for mixing the compressed product and the mixed olefin rich product to form a combined product. A flow line for feeding the knock-out liquid to the main fractionator may also be provided.

In another aspect, embodiments herein relate to a process for separating hydrocarbon mixtures. The separation process includes conveying a cracked reaction effluent comprising a mixture of hydrocarbons, including methane (C1) to heavy (C12+) hydrocarbons to a main fractionator, wherein the main fractionator receives the mixture of hydrocarbons and separates the mixture of hydrocarbons into an overhead vapor comprising C1 to C6+ hydrocarbons, a side draw liquid fraction, and a bottoms product fraction, and feeding the overhead vapor to a main fractionator overheads condensation and high-pressure separation system to partially condense the overheads vapor and compress the uncondensed vapor to produce a compressed gas fraction and a compressed liquids fraction. The compressed liquids fraction are fed to a demethanizer to separate the compressed liquids fraction into a demethanizer overheads vapor fraction, comprising methane rich gas, and a demethanizer bottoms fraction, comprising C2 to C6+ hydrocarbons. The demethanizer bottoms fraction are fed to a depentanizer to separate the demethanizer bottoms fraction into a depentanizer overheads fraction, comprising C2 to C5 olefins, and a depentanizer bottoms fraction, comprising C6+ hydrocarbons. The process also includes, in an adsorber, contacting, in countercurrent flow, a portion of the depentanizer bottoms fraction and the side draw liquid fraction with the compressed gas fraction to produce an absorber overheads fraction comprising methane and an absorber bottoms fraction.

In a further aspect, embodiments herein relate to a hydrocarbon separation process including conveying a cracked reaction effluent comprising a mixture of hydrocarbons, including methane (C1) to heavy (C12+) hydrocarbons to a main fractionator, wherein the main fractionator receives the mixture of hydrocarbons and separates the mixture of hydrocarbons into an overhead vapor comprising C1 to C6+ hydrocarbons, a side draw liquid fraction, and a bottoms product fraction. The overhead vapor is fed to a main fractionator overheads condensation and high-pressure separation system to partially condense the overheads vapor and compress the uncondensed vapor to produce a compressed gas fraction and a compressed liquids fraction. The compressed liquids fraction is fed to a first distillation column to separate the compressed liquids fraction into a first overheads vapor fraction and a first bottoms fraction. The process further includes feeding the first bottoms fraction to a second distillation column to separate the first bottoms fraction into a second overheads fraction and a second bottoms fraction. The process also includes, in an absorber, contacting, in countercurrent flow, a portion of the second bottoms fraction and the side draw liquid fraction with the compressed gas fraction to produce an absorber overheads fraction comprising offgas and an absorber bottoms fraction.

Other aspects and advantages will be apparent from the following description and the appended claims.

### BRIEF DESCRIPTION OF DRAWINGS

FIG. 1 illustrates a simplified process flow scheme for the product recovery section according to conventional industry practice (Prior Art).

FIGS. 2-5 illustrate simplified process flow schemes for a product recovery section according to one or more embodiments disclosed herein, where like numerals represent like parts. Embodiments herein consider combining absorption and integrates deethanizer and debutanizer sections to generate a high-quality product mix of C2 to C4 or C2 to C5 hydrocarbons and BTX (benzene, toluene, and mixed xylene) rich naphtha.

### DETAILED DESCRIPTION

Embodiments herein relate generally to conversion of heavier hydrocarbons to lighter hydrocarbons, such as by cracking or hydrocracking. Embodiments herein further relate to the separation and recovery of the resulting cracked hydrocarbon products.

As noted above, refiners may be shifting away from gasoline production in their FCC or RFCC units, producing a higher volume of lighter hydrocarbons, such as ethylene, propylene, and butylenes. For example, a cracking reaction system, such as a Single Regenerator Dual Catalyst (SRDC) or a combination of FCC/Residue FCC and SRDC, may be used to generate a cracked hydrocarbon effluent having a higher proportion of lighter hydrocarbons. The SRDC system and a combined FCC+SRDC system are describe in U.S. Pat. No. 10,758,883, among other offerings from Lummus Technology LLC.

The effluent resulting from processing of the feed in a SRDC or combined FCC+SRDC may then be fed to separation systems according to embodiments herein for separation and recovery of the various products.

Hydrocarbon feeds to cracking reactors, including SRDC, RFCC, and FCC reactors, may include any number of hydrocarbons, hydrocarbon cuts, or hydrocarbon mixtures or intermediates. Feedstocks may include typical Fluid Catalytic Cracking (FCC) feedstocks, such as gas oils, but feedstocks for producing a cracked product herein may range in carbon number C2 or greater. Hydrocarbon mixtures useful in embodiments disclosed herein may include various hydrocarbon mixtures, including those having a boiling point range, where the end boiling point of the mixture may be less than 450° C. such as cracked naphtha from FCC units or any olefinic stream from a refinery. In some cases, it can also be greater than 450° C., depending upon source and type of the feedstock. Various systems incorporating SRDC may also process whole crudes or condensates to produce the cracked effluent fed to separation systems according to embodiments herein. In summary, processes disclosed herein can be applied to crudes, condensates and hydrocarbon mixtures with a wide boiling curve and end points. Such hydrocarbon mixtures may include whole crudes, virgin crudes, hydroprocessed crudes, gas oils, vacuum gas oils, heating oils, jet fuels, diesels, kerosenes, gasolines, synthetic naphthas, raffinate reformates, Fischer-Tropsch liquids, Fischer-Tropsch gases, natural gasolines, distillates, virgin naphtha, cracked naphtha, natural gas condensates, atmospheric pipestill bottoms, vacuum pipestill streams including bottoms, wide boiling range naphtha to gas oil condensates; heavy non-virgin hydrocarbon streams from refineries, vacuum gas oils, heavy gas oils, atmospheric residuum, hydrocracker wax, and Fischer-Tropsch wax, among others. In some embodiments, the hydrocarbon mixture may include hydrocarbons boiling from the naphtha range or lighter to the vacuum gas oil range or heavier. If desired, these feeds may be pre-processed to remove a portion of the sulfur, nitrogen, metals, and Conradson Carbon upstream of processes disclosed herein.

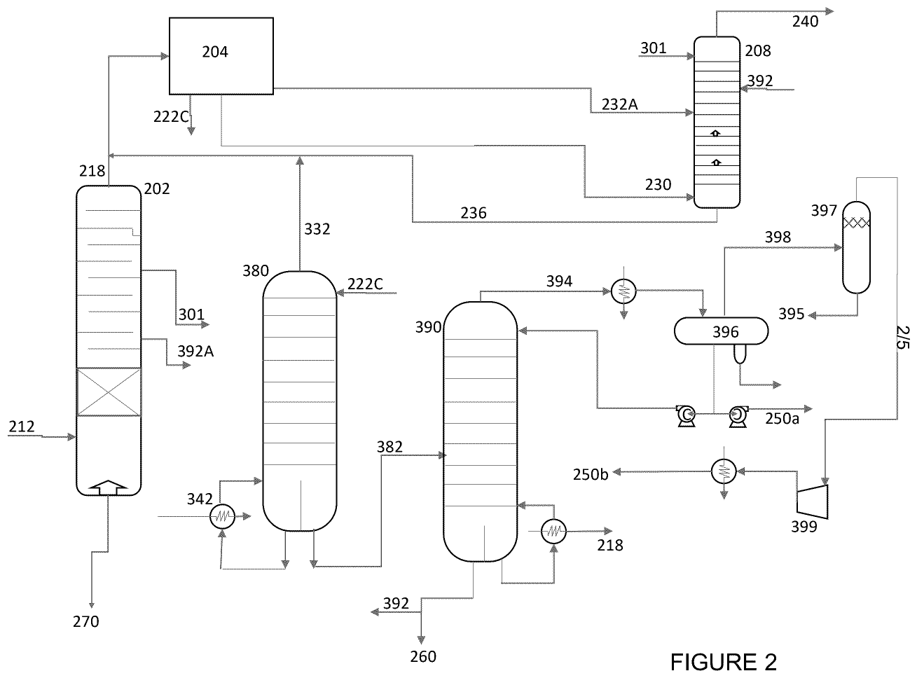

Following generation of a cracked reaction product in an upstream cracking reaction system, the reaction effluent is fed to a separation system according to embodiments herein, such as illustrated in FIG. 2.

FIG. 2 illustrates a simplified process flow diagram of separation systems according to embodiments herein. The separation system includes main fractionator 202, fractionator overheads and high-pressure separation system 204, absorber 208, and a fractionation zone including a demethanizer 380 and a debutanizer/depentanizer 390. Primary products recovered from the separation system may include methane rich fraction 240, a mixture of C2-C5 or C2-C4 fraction 250a and/or 250b, naphtha range fraction 260, and heavy product fraction 270. Other fractions may be recovered as well, such as various intermediate hydrocarbon cuts (not illustrated) recovered from main fractionator 202, among others.

A cracked reaction effluent 212 is fed to the main fractionator 202 for separating the cracked reactor effluent into bottoms fraction (heavy product fraction) 270, one or more side draw liquid fractions 301, and overheads vapor 218. The side draw liquid fraction, or a portion thereof, is fed to absorber 208, while the overheads vapor 218 is processed through the main fractionator overheads and the high-pressure separation system 204. Side draw liquid fraction 301 may be withdrawn from the main fractionator at a column height to provide a side draw fraction that contains primarily naphtha range hydrocarbons, although some heavier (such as light cycle oil) and lighter components may also be present. It is also possible to side draw one or more heavier products 392A, such as light cycle oil and/or heavy cycle oil, from the middle section of main fractionator 202.

The main fractionator overheads and high-pressure compression system 204 is used to partially condense and compress overheads vapor 218 to produce overheads liquid fraction 232, overheads compressed vapor fraction 230 and overheads compressed liquid fractions 222C. A portion 232B of overheads liquid fraction 232 is fed to the main fractionator as a reflux, and a portion 232A may be fed to absorber 209. Compressed liquid fraction 222C is fed as reflux to demethanizer 380. Compressed gas fraction 230 is fed to absorber 208.

Absorber 208 receives and contacts in countercurrent flow the side draw liquid fraction 301 and the overhead compressed gas fraction 230. The heavier hydrocarbons contained in the side draw 301 as well as in the liquid fraction 232A from main fractionator overhead and high-pressure system 204, as well as a portion of stabilized naphtha range material, lean oil 392 from fractionation (debutanizer/depentanizer) zone 390, are also fed to absorber 208 for absorption of the ethane and heavier hydrocarbons. The liquid from absorber 208 is recovered as an absorber bottoms fraction 236. The methane and any other light gases present (such as hydrogen, H2S, nitrogen, carbon dioxide, etc.), are recovered from the absorber 208 as methane rich product fraction 240. Methane rich product fraction 240 may be recovered as an offgas in some embodiments and may be processed in a gas treatment plant to recover the methane.

Compressed liquid fraction 222C from high-pressure separation system 204 is fed to a fractionation zone for separation and recovery of the ethylene and ethane rich material and heavier hydrocarbons in desired hydrocarbon cuts. As illustrated in FIG. 2, the fractionation zone may include one or more distillation columns 380, 390 configured to separate the compressed liquid fraction 222C into an overheads vapor fraction 332, comprising methane rich gas, light olefin rich fractions 250a and 250b, such as C2 to C4or C2 to C5 hydrocarbon product fractions, and bottoms fraction 260, comprising C5+ or C6+ naphtha (BTX rich) range hydrocarbons.

Lights fraction 332 and absorber bottom liquid 236 are fed to the fractionator overhead and high-pressure separation system 204 for further recovery of C2+ components and eventual separation of the methane in absorber 208. Bottoms fraction 260, such as naphtha range hydrocarbons from fractionation zone 380, as well as heavy product fraction 270 may be recirculated, for example, to the cracking reactors for additional production of lighter C2-C5 range olefins or meeting the endothermic heat balance requirements.

As outlined briefly above with respect to FIG. 2, the separation system provides a method for efficient and cost-effective separation of cracked hydrocarbon effluents, such as from FCC, RFCC, or SRDC reactor systems, or combined product vapors from various cracking reactors, into a light olefin rich product blend. The resulting product blend rich in light olefins (C2 to C5 petrochemical building blocks) then may be separated into premium products such as ethylene, propylene, butylene, C5 olefins and aromatic rich naphtha products. Such separation into premium products may be performed on site or the product blend may be transported to a central facility for processing into the premium products.

SRDC or light olefins FCC/RFCC or combination of SRDC+FCC/RFCC reactor systems typically produce lighter molecules (C2, C3, C4 and C5) and BTX (benzene, toluene and mixed xylenes) rich naphtha at increased volumes as compared to the typical gasoline mode FCC units. As the traditional FCC/RFCC units shift away from gasoline production, toward lighter molecules and BTX rich naphtha, the commonly used FCC separation units may be unsatisfactory for processing the reactor effluents to end user products. Separations geared for a heavier product mixture and gasoline recovery would not perform satisfactory with an SRDC or any light olefins FCC/RFCC product vapor or other cracked products rich in lighter olefin molecules and BTX rich naphtha. Separation systems according to embodiments herein, however, can process such cracked effluents to produce a blend of light olefin rich C2-C4 or C2-C5 products as a single product stream. This may reduce the burden for refiners in terms of capital costs, operating costs, and plot area, while providing no requirement for finished products or gasoline destruction. The reduction in gasoline in the refinery and production of higher quantities of light olefins may make refineries more profitable during decreased gasoline demand periods.

FIG. 2, as described above, is a simplified process flow diagram of separation systems according to embodiments herein. Embodiments of separation systems herein may also provide for decreasing the absorbent (stabilized naphtha/gasoline) recirculation between the primary absorber and downstream fractionation units. This helps in reducing the size of several pieces of equipment and utility requirements.

As noted above, absorber 208 may receive each of the side draw 301 and the compressed vapors 230, which may include methane and C2-C4 olefins, among other components. The compressed vapors may be fed to a lower portion of the absorber, while the side draw 301 may be fed to an upper portion of the absorber, thus contacting the lighter hydrocarbons in countercurrent flow with the heavier hydrocarbons, absorbing the ethylene and other olefins and hydrocarbons contained in the compressed vapors produced in the overheads processing. The absorber may also receive, as additional absorbents, overheads condensate fraction 232A, received from system 204, as well as main absorbent, the portion 392 of the depentanizer/debutanizer 390 bottoms fraction not recovered as a naphtha product fraction 260. The countercurrent contact of the compressed vapors with the multiple absorbents may provide for efficient recovery of the C2 and heavier hydrocarbons, producing an absorber overheads fraction 240 that contains methane rich gas and no or a minimal content of hydrocarbons heavier than methane. The absorbent and the absorbed hydrocarbons may be recovered as an absorber bottoms fraction 236, which may be recycled back to main fractionator 202 overhead and high-pressure separation system 204, to finally be reprocessed in the fractionation zone 380, 390. Additional temperature control of the absorber may be provided by use of one or more interstage cooling loops, withdrawing, cooling, and returning liquids to the absorber 208.

The fractionation zone may include, as primary separators, a demethanizer 380 and a depentanizer/debutanizer 390. Demethanizer 380 may receive compressed liquid fraction 222C from main fractionator 202, separating the hydrocarbons therein into a light overhead fraction 332, which may be primarily methane and some light olefins, and a bottoms fraction 382, which may include C2 and heavier hydrocarbons present in the compressed liquid fraction 232. Reboiler 342 may exchange heat between a portion of a main fractionator side draw (not illustrated in FIG. 2) and a demethanizer bottoms reboiler draw to provide reboiler vapors to demethanizer 380.

Bottoms fraction 382 may be fed to depentanizer/debutanizer 390 for separation of the C2+ hydrocarbons. As the name implies, when operated as a depentanizer, unit 390 may be operated to recover C5 and lighter components as overheads draw 394 and C6+ hydrocarbons as bottoms draw 392. Similarly, when operated as a debutanizer, unit 390 may be operated to recover C4 and lighter components as overheads draw 394 and C5+ hydrocarbons as bottoms draw 392. Depentanizer/Debutanizer overheads 394 may then be partially condensed (if required using a refrigeration or chilled water system) and recovered in a reflux drum 396. The condensed C2-C5 or C2-C4 hydrocarbon liquids may be used to reflux the depentanizer/debutanizer 390 and a remaining portion of the C2-C5 or C2-C4 hydrocarbon liquids may be recovered as a first light product stream 250a. The C2-C5 or C2-C4 vapors 398 recovered from reflux drum 396 may be processed in a knockout drum 397 to recover any entrained liquids 395, the vapors then being compressed via a compressor 399 and recovered as a second light olefin rich product 250b. The first light olefins rich product can also be fed to compressor 399 for making light olefins rich product (C2 to C4 or C2 to C5) that can be transported to a centralized location for further product recovery. Alternatively, first light olefins rich product stream 250a can combined with second light olefin rich product 250b downstream of compressor 399, making a combined light olefins rich product (C2 to C4 or C2 to C5) that can be transported to a centralized location for further product recovery. The bottoms draw from debutanizer/depentanizer 390 may be cooled, and a portion of the C6+ or C5+ hydrocarbons may be recovered as a naphtha product 260, while remaining portion 392 of the bottoms draw may be returned to absorber 208. As described above, C2-C5 or C2-C4 product streams 250a, 250b may be combined and recovered as a single product stream (not illustrated).

The main fractionator, absorber, demethanizer, and debutanizer/depentanizer may be integrated with respect to flow and heat. Integration of the units in this or a similar manner may thus provide for efficient operations and separation of the cracked hydrocarbon reactor effluents into a C2-C4 or C2-C5 olefin containing product stream and one or more heavier streams, such as a naphtha stream and a heavy hydrocarbon fraction. Each of the heavier streams may be recovered as a product or may be returned to the cracking zone for continued production of light olefins.

In some embodiments, distillation column 380 may be sized and configured such that it may be operated flexibly as either demethanizer or deethanizer, recovering C1 or C1-C2 hydrocarbons as overheads 332, respectively. Further, in some embodiments, distillation column 390 may be sized and configured such that it may be operated flexibly as either depentanizer or debutanizer, recovering C2-C5 or C2-C4 hydrocarbons as overheads 394 (or recovering C3-C5 or C3-C4 hydrocarbons when column 380 is operating as a deethanizer). Operating flexibility in demethanizer 380 depends on methane gas mixture quality (mainly heating value) produced from absorber 208, also on the demand of ethylene, which can be extracted from C2-C5 mixture separated from depentanizer 390. Operating column 380 as a deethanizer will add up marginally in operating cost. Similarly, operating as a debutanizer instead of a depentanizer will add up marginally in operating cost. Operating mode largely depends on C5 demand. Flexible operations, according to embodiments herein, may include operating in a first mode of operation for a period of time, followed by or preceded by operating in a second mode of production for a period of time.

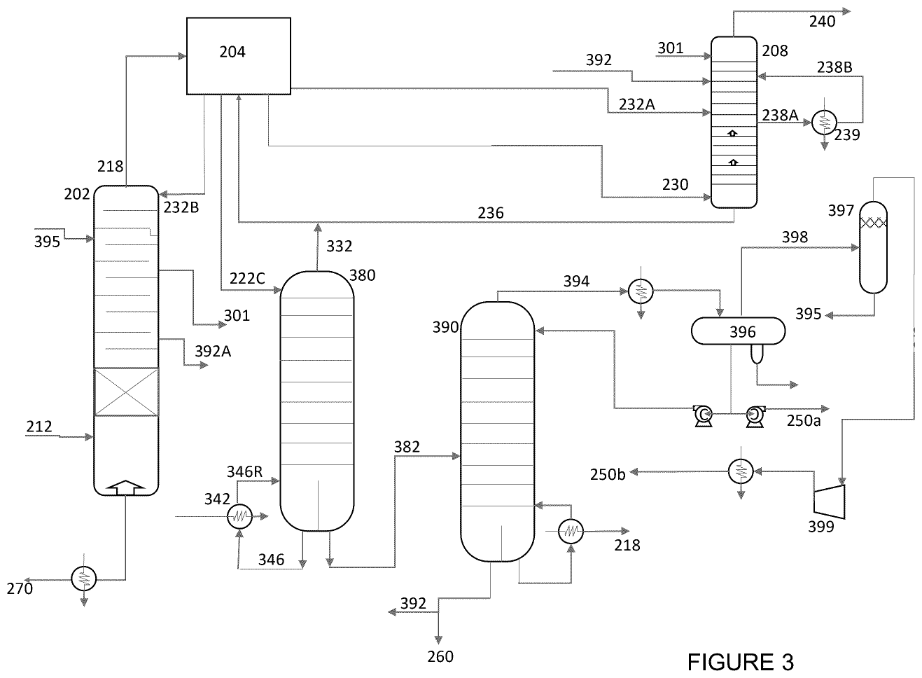

In some embodiments, such as illustrated in FIG. 3, the hydrocarbon separation system may include a flow line 232A for feeding a portion of the condensate liquids fraction to the absorber, and a flow line 232B may be provided for feeding a second portion of the condensate liquids as a reflux to the main fractionator. A flow line 222C may be provided for feeding the compressed liquids fraction to the first distillation column.

As also illustrated in FIG. 3, the absorber 208 may be further configured to produce an absorber side draw fraction 238A and to receive a cooled absorber side draw fraction 238B; while only one is illustrated, multiple side draws and cooled returns may be provided. The system may further include a heat exchanger 239 configured to cool the absorber side draw fraction 238A and to produce the cooled absorber side draw fraction 238B.

As further illustrated in FIG. 3, in some embodiments the system may additionally include a flow line for feeding the knock-out liquid 395 from knock-out drum 397 to main fractionator 202.

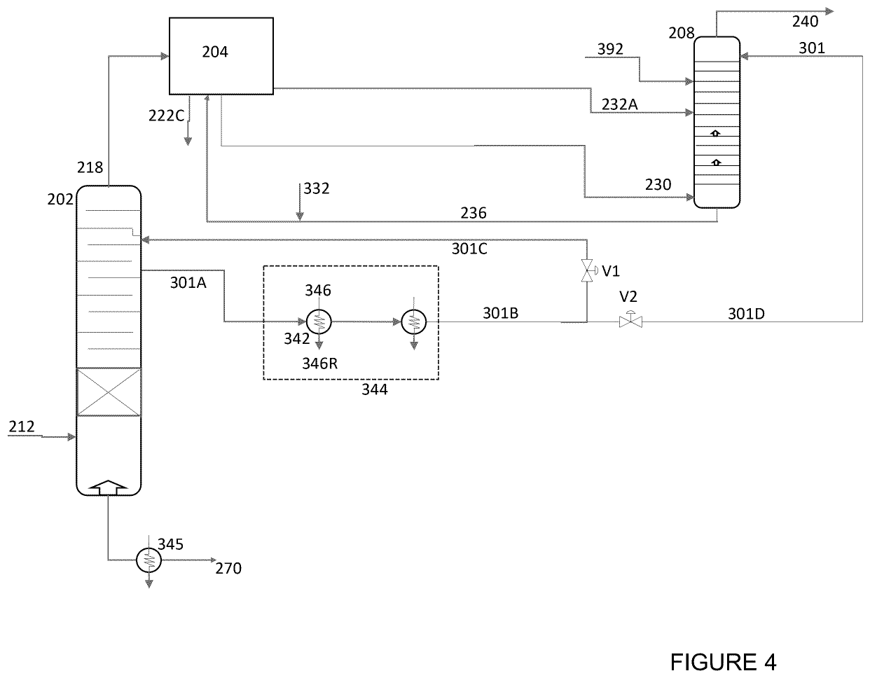

As mentioned above, various methods may be used to integrate heat requirements of the system. A couple options for heat integration are illustrated in FIG. 4. In some embodiments, the system may include a heat exchange system 344, disposed downstream of main fractionator 202 and upstream of absorber 208. The heat exchange system may include one or more heat exchangers configured to cool the main fractionator side draw 301A and to produce a cooled main fractionator side draw 301B fed to absorber 208. In some embodiments, the resulting cooled stream may be returned from the heat exchange system as a cooled return to the main fractionator, and such may be applied on one or more side draws.

As also illustrated in FIG. 4, the system may include a flow control system, such as including flow control valves V1 and V2, configured for diverting a portion 301C of the cooled main fractionator side draw 301B to main fractionator 202 as a reflux and for feeding a remaining portion 301D of the cooled main fractionator side draw 301B to absorber 208.

As further illustrated in FIG. 4, the system may also include a second heat exchange system 345, including one or more heat exchangers for cooling the main fractionator bottoms fraction 270.

In some embodiments, the heat exchange system 344 and the second heat exchange system 345 are configured to heat water via the main fractionator side draw 301A and the main fractionator bottoms fraction 270 to produce a medium pressure steam stream (15-150 psig) or a high-pressure steam stream (150-1000 psig).

Referring collectively to FIGS. 3 and 4, in some embodiments, at least one of the two or more heat exchangers of the heat exchange system 344 include a heat exchanger 342 configured to reboil a bottoms stream 346 recovered from demethanizer 380 and to return a heated reboil bottoms stream 346R to the demethanizer. For systems including more than one reboiler 342, additional reboiler vapor 346R may be provided via heating of a bottoms draw 346 via indirect heat exchange against entrained liquids 395 during transport from knock out drum 397 to main fractionator 202.

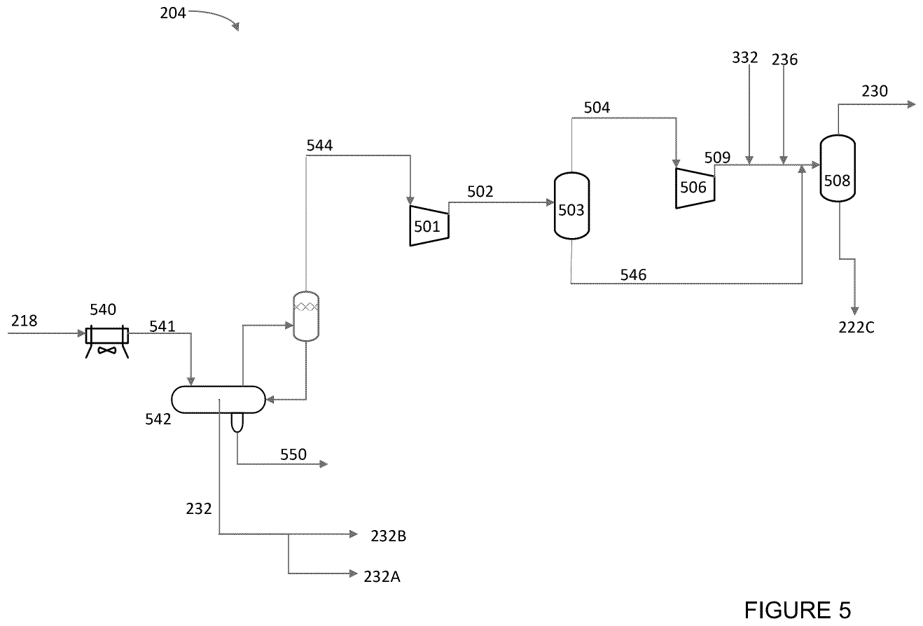

Referring now to FIG. 5, one embodiment of the main fractionator overheads and high-pressure separation system 204 is illustrated. The high-pressure separation system 204 may include a heat exchanger 540 to partially condense overhead vapor 218 received from main fractionator 202, and the partially condensed overheads 541 may be separated in a condensate drum 542 to provide condensate liquids 232 and an overheads vapor fraction 544. Condensate liquids 232 may then be divided to provide reflux 232B to main fractionator 202 and feed 232A to absorber 208. A first stage compressor 501 is provided to compress the overheads vapor fraction 544 and produce a compressed overheads vapor fraction 502 fed to separator 503. Separator 503 may then be used to separate any condensates from the compressed overheads vapor fraction, producing a compressed vapor fraction 504 and a compressed liquid fraction 546. A second stage compressor 506 may be provided to further compress the compressed vapor fraction 504 and to produce a second stage compressor effluent 506. A high-pressure separator 508 may then be used to separate liquids and vapors contained in the second stage compressor effluent 509 and compressed liquid fraction 546 to produce a high-pressure separation system liquid stream 222C and a high-pressure separation system vapor stream 230. A sour water stream 550 may also be produced.

In some embodiments, a mixing system may be provided to mix the second stage compressor effluent 509 with the absorber bottoms stream 236 and the demethanizer overheads vapor fraction 332. Such mixing may occur upstream of or within separator 508.

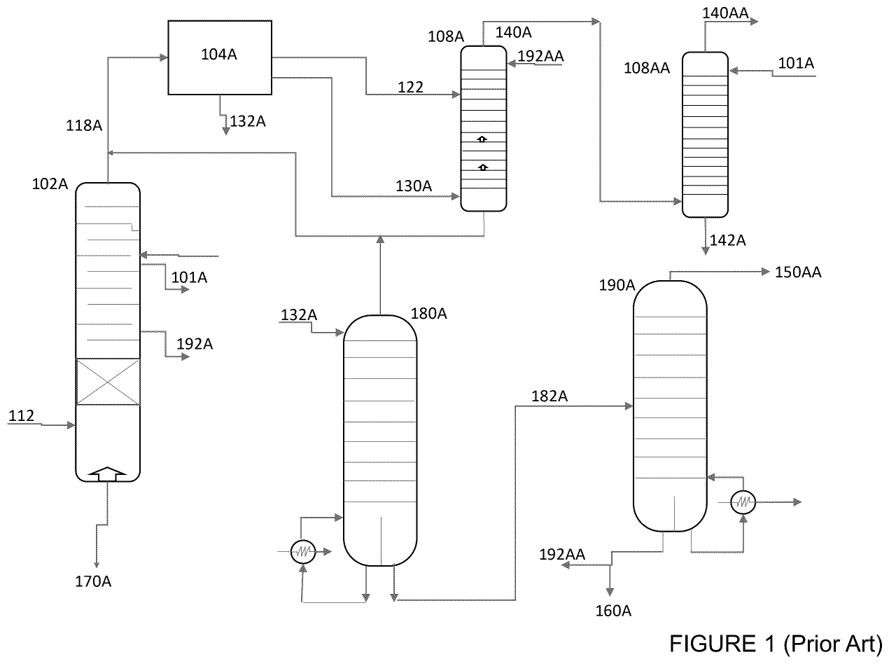

Operating conditions at some sections of separation schemes according to embodiments herein may widely be different from the conventional separation scheme of FIG. 1. Operating conditions are to be set based on the properties and product requirement on a case-to-case basis. However, operating conditions of each section are summarized below.

Main Fractionator: Depending on the feed quality, rate and type and design requirement, main fractionator 202 may be equipped with trays, packing or combination. Main fractionator top temperature controls the naphtha end boiling point, which may vary based on feed quality and recovery targets. Main fractionator top temperature may vary from 90-160° C., while bottoms temperature may vary from 190-350° C., which controls the bottom fraction initial boiling point. Operating pressure typically depends on yields required from upstream reactor(s) based on feed, which may vary the top pressure from as low as 0.2 kg/cm² (g) to as high 3.0 kg/cm² (g) (where (g) indicates gauge pressure).

Absorber: Absorber 208 operating conditions may be optimized to achieve the desired recovery of light hydrocarbons. Depending on yield, recovery, utility system, top operating pressure may vary from 9-20 kg/cm² (g), and temperature may be 15-60° C.

Demethanizer: Demethanizer 380 operating conditions may be optimized to achieved the maximum recovery of C2 components, and, depending on C2 recovery utility system, the top operating pressure may vary from 10 kg/cm² (g) to 20 kg/cm² (g) and bottom operating temperature may vary from 30-120° C.

Demethanizer as Deethanizer: As explained above, there is complete flexibility to operate demethanizer 380 as a deethanizer, depending on methane gas mixture specification and ethylene demand. When operated as a deethanizer, top operating pressure may vary from 9 kg/cm² (g) to 20 kg/cm² (g) and bottom temperature may vary from 70-150° C.

Depentanizer: Depentanizer 390 bottom temperature depends on C6+ mixture end point, which is indirectly controlled by main fractionator 202 top temperature. Operating pressure for depentanizer top depends on utility system available and may vary from 4.5 kg/cm² (g) to 12 kg/cm² (g) and bottom temperature may vary from 160-280° C. Higher bottom temperature in depentanizer may increase the fouling in the reboiler.

Depentanizer as Debutanizer: As explained above, there is complete flexibility to operate depentanizer 390 as a debutanizer, depending on C5 demand. Again, debutanizer bottom temperature is indirectly controlled by main fraction top temperature, which fixes the C6+ mixture end point. Operating pressure of debutanizer top may vary from 9-16kg/cm² (g) depending on availability of utility system and bottom temperature may vary from 160-280° C.

The following table illustrates the benefits of embodiments disclosed herein. In particular, the C2's recovered quite efficiently in blended light olefin rich product (O-grade blend) while minimizing the capital and operating cost.

| | | Conventional | C2s recovery with C2-C5 | C2s recovery with C2-C4 |
|---|---|---|---|---|
| Parameters | Unit | Gas plant | product range per FIG. 2 | product range per FIG. 2 |
| C2 Product | kg/h | 0 | 2979 | 3020 |
| C3 product | kg/h | 30077 | 30086 | 30092 |
| C4 Product | kg/h | 19835 | 20476 | 19970 |
| C5 Product | kg/h | 0 | 11871 | 0 |
| Total O-Grade Blend | kg/h | 49912 | 65411 | 53082 |
| Naphtha Product | kg/h | 97201 | 85382 | 96915 |
| Offgas heating value | kcal/kg | 7,920 | 7,132 | 7,143 |
| | | Conventional | C2s recovery with C2-C5 | C2s recovery with C2-C4 |
| Parameters | Unit | Gas plant | product range per FIG. 2 | product range per FIG. 2 |
| Cold Water | m³/h | 5958 | 3652 | 3695 |
| consumption | | | | |
| High pressure steam | Gcal/h | 18.2 | 11.0 | 12.0 |
| consumption | kg/h | 44688 | 27188 | 29512 |
| Medium pressure | Gcal/h | 21.1 | 21.1 | 21.1 |
| steam production | kg/h | 38347 | 41163 | 41163 |
| Power | KW | 4163 | 4632 | 4319 |

As noted above, separation systems herein may be operated at various conditions, depending upon the cracked reaction effluent being fed to the system, as well as the desired product separations (demethanizer vs, deethanizer, debutanizer vs. depentanizer. In some embodiments, product streams produced using separation units herein may meet the following specifications:
C2-C5 product stream: C1<3.0 wt %, C2>2.0 wt %, C6+=traces;
C2-C4 product stream: C1<3.0 wt %, C2>2.0 wt %, C5+=traces;
   Naphtha Depentanizer/Debutanizer bottoms:
TBP Range: C6-250° C., RVP<4 kPa (Depentanizer),
TBP range: C5-250° C. (Debutanizer), RVP<8.0 kPa (Debutanizer)
   Main fractionator bottoms: TBP 5 Vol %>190° C.

As described above, embodiments herein may provide various benefits as compared to traditional separations of FCC cracked effluents targeting gasoline production. The main benefits of the embodiments herein may include one or more of the following: (a) Embodiments disclosed herein separate the reaction effluent in cost effective way with overall less operating and capital cost as compared to current conventional industry practice. (b) Separation scheme proposed is also less energy intensive and require lesser plot space due to less number or smaller equipment required for separation. (b) As electric vehicles gain momentum, which reducing drastically the gasoline demand, conventional or prior art separation method will not give the desired economical results. Hence, the scheme of embodiments herein provides an opportunity to creatively re-configure the process scheme to process much higher amounts of light olefins rich products with minimal changes to the existing equipment, as for example convert existing deethanizer to demethanizer. Such schemes help substantially reduce the lean oil recirculation and hence allow processing higher quantities of light olefins rich products with minimal modifications to the existing FCC gas plant. (d) Embodiments herein also minimize the utilities consumption and help reduce the capital, operating costs and plot space of a refinery complex. (e) The off-gas product generated from embodiments herein is still good in heating/calorific value and can be blended refinery fuel gas system for meeting the internal fuel requirements.

Unless defined otherwise, all technical and scientific terms used have the same meaning as commonly understood by one of ordinary skill in the art to which these systems, apparatuses, methods, processes and compositions belong.

The singular forms “a,” “an,” and “the” include plural referents, unless the context clearly dictates otherwise.

As used here and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

When the word “approximately” or “about” are used, this term may mean that there can be a variance in value of up to ±10%, of up to 5%, of up to 2%, of up to 1%, of up to 0.5%, of up to 0.1%, or up to 0.01%.

Ranges may be expressed as from about one particular value to about another particular value, inclusive. When such a range is expressed, it is to be understood that another embodiment is from the one particular value to the other particular value, along with all particular values and combinations thereof within the range.

“Mixing systems,” “mixers” or like terms as used herein refer to flow lines or mixing tees, which may or may not include static mixers, as well as, pumps, vessels, or agitated vessels, or other equipment known in the art that may be used to combine and blend two or more flow streams.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

What is claimed as new and desired to be protected by Letters Patent is:

1. A hydrocarbon separation system comprising:
a flow line for conveying a cracked reaction effluent comprising a mixture of hydrocarbons, including methane (C1) to heavy (C12+) hydrocarbons, from one or more cracking reactors;
a main fractionator, wherein the main fractionator is configured to receive the mixture of hydrocarbons, and wherein the main fractionator is configured to separate the mixture of hydrocarbons into an overhead vapor comprising C1 to C6+ hydrocarbons, a side draw liquid fraction, and a bottoms product fraction;
a main fractionator overheads condensation and high-pressure separation system configured to partially condense the overheads vapor and compress the uncondensed vapor to produce a compressed gas fraction and a compressed liquids fraction;
a first distillation column configured to receive the compressed liquids fraction and to separate the compressed liquids fraction into a first overheads vapor fraction and a first bottoms fraction; and
a second distillation column configured to receive the first bottoms fraction and to separate the first bottoms fraction into a second overheads fraction and a second bottoms fraction;
an absorber configured to receive and contact in countercurrent flow a portion of the second bottoms fraction and the side draw liquid fraction with the compressed gas fraction, and to produce an absorber overheads fraction comprising offgas and an absorber bottoms fraction.

2. The hydrocarbon separation system of claim 1, wherein:
the first distillation column is sized and configured to operate flexibly as a demethanizer in a first mode of operation and as a deethanizer in a second mode of operation;
the absorber is sized and configured to operate flexibly to recover an offgas comprising methane and essentially no C2 hydrocarbons in the first mode of operation and to recover an offgas comprising methane and C2 hydrocarbons in the second mode of operation.

3. The hydrocarbon separation system of claim 1, wherein the second distillation column is sized and configured to operate flexibly as a debutanizer in a first mode of operation and as a depentanizer in a second mode of operation.

4. The hydrocarbon separation system of claim 1, further comprising one or both of:
a flow line for feeding the absorber bottoms fraction to the main fractionator overheads condensation and high-pressure separation system; and
a flow line for feeding the first overheads vapor fraction to the main fractionator overheads condensation and high-pressure separation system.

5. The hydrocarbon separation system of claim 1, wherein the compressed liquid fraction comprises a first compressed liquids fraction comprising C4 to C6+ hydrocarbons, and a second compressed liquids fraction comprising C1 to C6+ hydrocarbons, and wherein the hydrocarbon separation system further comprises:
a flow line for feeding a portion of the first compressed liquids fraction to the absorber;
a flow line for feeding a second portion of the compressed liquids fraction as a reflux to the main fractionator; and
a flow line for feeding the second compressed liquids fraction to the first distillation column.

6. The system of claim 1, wherein the absorber is further configured to produce an absorber side draw fraction and to receive a cooled absorber side draw fraction, the system further comprising a heat exchanger configured to cool the absorber side draw fractions and to produce the cooled absorber side draw fraction.

7. The system of claim 1, further comprising:
a heat exchange system, disposed downstream of the main fractionator and upstream of the absorber, the heat exchange system comprising two or more heat exchangers configured to cool the main fractionator side draw and to produce a cooled main fractionator side draw fed to the absorber; and
a flow control system configured for diverting a portion of the cooled main fractionator side draw to the main fractionator as a reflux and for feeding a remaining portion of the cooled main fractionator side draw to the absorber.

8. The system of claim 7, further comprising a second heat exchange system for cooling the main fractionator bottoms fraction, wherein the heat exchange system and the second heat exchange system are configured to heat water via the main fractionator side draw and the main fractionator bottoms fraction to produce a medium or high-pressure steam stream.

9. The system of claim 7, wherein at least one of the two or more heat exchangers of the heat exchange system comprises a heat exchanger configured to reboil a bottoms stream recovered from the first distillation column and to return a heated reboil bottoms stream to the first distillation column.

10. The system of claim 1, wherein the high-pressure separation system comprises:
a heat exchanger and condensate drum for partially condensing the overheads vapor to produce a first liquid fraction and an overhead vapor fraction;
a first stage compressor configured to compress the overheads vapor fraction and produce a compressed overheads vapor fraction;
a separator configured to separate condensates from the compressed overheads vapor fraction and to produce a compressed vapor fraction and a compressed liquid fraction;
a second stage compressor configured to further compress the compressed vapor fraction and to produce a second stage compressor effluent;
a high-pressure separator configured to separate liquids and vapors contained in the second stage compressor effluent and the compressed liquid fraction to produce a high-pressure separation system liquid stream and the compressed gas fraction; and
a mixing system configured to mix the second stage compressor effluent with the absorber bottoms stream and the first distillation column overheads vapor fraction upstream of the high-pressure separator.

11. The system of claim 1, further comprising a second distillation column overheads system, the second distillation column overheads system comprising:
a heat exchanger for partially condensing the second distillation column overheads fraction;
a drum for separating the partially condensed second distillation column overheads fraction into a second distillation column overheads liquid fraction and a second distillation column overheads vapor fraction.
a knock-out drum to collect any liquids entrained in the second distillation column overheads vapor fraction, producing a knock-out vapor and a knockout liquid;
a compressor for compressing the knock-out vapor, producing a compressed product;
a flow line for feeding a portion of the second distillation column overheads liquid fraction to the second distillation column as a reflux; and
a flow line for recovering a remaining portion of the second distillation column overheads liquid fraction as a mixed olefin rich product.

12. The system of claim 11, further comprising one or both of:
a mixing system for mixing the compressed product and the mixed olefin rich product to form a combined product; and
a flow line for feeding the knock-out liquid to the main fractionator.

13. The system of claim 1, further comprising a reaction system comprising the one or more cracking reactors, and wherein the cracking reactors are configured to produce as a target reaction product C2 to C5 olefins and aromatics comprising benzene, toluene, and mixed xylenes.

14. A hydrocarbon separation process comprising:
conveying a cracked reaction effluent comprising a mixture of hydrocarbons, including methane (C1) to heavy (C12+) hydrocarbons to a main fractionator, wherein the main fractionator receives the mixture of hydrocarbons and separates the mixture of hydrocarbons into an overhead vapor comprising C1 to C6+ hydrocarbons, a side draw liquid fraction, and a bottoms product fraction;
feeding the overhead vapor to a main fractionator overheads condensation and high-pressure separation system to partially condense the overheads vapor and compress the uncondensed vapor to produce a compressed gas fraction and a compressed liquids fraction;
feeding the compressed liquids fraction to a first distillation column to separate the compressed liquids fraction into a first overheads vapor fraction and a first bottoms fraction;
feeding the first bottoms fraction to a second distillation column to separate the first bottoms fraction into a second overheads fraction and a second bottoms fraction; and
in an absorber, contacting, in countercurrent flow, a portion of the second bottoms fraction and the side draw liquid fraction with the compressed gas fraction to produce an absorber overheads fraction comprising offgas and an absorber bottoms fraction.

15. The hydrocarbon separation process of claim 14, further comprising:
operating the first distillation column as a demethanizer for a first period of time and as a deethanizer for a second period of time; and
operating the absorber to recover an offgas comprising methane and essentially no C2 hydrocarbons during the first period of time, and to recover an offgas comprising methane and C2 hydrocarbons during the second period of time.

16. The hydrocarbon separation process of claim 14, wherein the second distillation column is sized and configured to operate flexibly as a debutanizer in a first mode of operation and as a depentanizer in a second mode of operation.

17. The hydrocarbon separation process of claim 14, further comprising one or both of:
feeding the absorber bottoms fraction to the main fractionator overheads condensation and high-pressure separation system; and
feeding the first overheads vapor fraction to the main fractionator overheads condensation and high-pressure separation system.

18. The hydrocarbon separation process of claim 14, wherein the compressed liquid fraction comprises a first compressed liquids fraction comprising C4 to C6+ hydrocarbons, and a second compressed liquids fraction comprising C1 to C6+ hydrocarbons, the process further comprising feeding a first portion of the first compressed liquids fraction to the absorber, feeding the second compressed liquids fraction to the first distillation column, and feeding a second portion of the first compressed liquids fraction as a reflux to the main fractionator.

19. The process of claim 14, wherein the absorber is further configured to produce an absorber side draw fraction and to receive a cooled absorber side draw fraction, the process further comprising cooling the absorber side draw fractions to produce the cooled absorber side draw fraction.

20. The process of claim 14, further comprising:
cooling, in a heat exchange system disposed downstream of the main fractionator and upstream of the absorber, the heat exchange system comprising two or more heat exchangers, the main fractionator side draw to produce a cooled main fractionator side draw fed to the absorber;
diverting a portion of the cooled main fractionator side draw to the main fractionator as a reflux and feeding a remaining portion of the cooled main fractionator side draw to the absorber; and
cooling, in a second heat exchange system, the main fractionator bottoms fraction.

21. The process of claim 20, further comprising one or both of:
heating water via the main fractionator side draw and the main fractionator bottoms fraction to produce a medium or high-pressure steam stream; and
reboiling a bottoms stream recovered from the first distillation column, wherein at least one of the two or more heat exchangers of the heat exchange system comprises a heat exchanger configured to reboil a bottoms stream recovered from the first distillation column and to return a heated reboil bottoms stream to the first distillation column.

22. The process of claim 14, wherein operation of the high-pressure separation system comprises:
cooling and partially condensing the overheads vapor to produce a first liquid fraction and an overhead vapor fraction;
compressing, in a first stage compressor, the overheads vapor fraction and produce a compressed overheads vapor fraction;
separating condensates from the compressed overheads vapor fraction to produce a compressed vapor fraction and a compressed liquid fraction;
compressing, in a second stage compressor, the compressed vapor fraction to produce a second stage compressor effluent;
separating liquids and vapors contained in the second stage compressor effluent to produce the compressed liquids fraction fed to the first distillation column and the compressed gas fraction fed to the absorber; and
mixing the second stage compressor effluent with the absorber bottoms stream and the first distillation column overheads vapor fraction.

23. The process of claim 14, further comprising, in a second distillation column overheads system:
partially condensing the second distillation column overheads fraction;
separating the partially condensed second distillation column overheads fraction into a second distillation column overheads liquid fraction and a second distillation column overheads vapor fraction.
collecting any liquids entrained in the second distillation column overheads vapor fraction, producing a knock-out vapor and a knock out liquid;
compressing the knock-out liquid, producing a compressed product;
feeding a portion of the second distillation column overheads liquid fraction to the second distillation column as a reflux; and
recovering a remaining portion of the second distillation column overheads liquid fraction as a mixed olefin rich product.

24. The process of claim 23, further comprising one or both of:
mixing the compressed product and the mixed olefin rich product to form a combined product; and
feeding the knock-out liquid to the main fractionator.

25. The process of claim 14, further comprising cracking a hydrocarbon feedstock in a reaction system comprising one or more cracking reactors to produce the cracked reaction effluent, wherein the one or more cracking reactors produce as a target reaction product C2 to C5 olefins and aromatics comprising benzene, toluene, and mixed xylenes.
